(19) United States
(12) Patent Application Publication
(10) Pub. No.: US 2015/0322479 A1
(43) Pub. Date: Nov. 12, 2015

(54) APPARATUS AND METHOD FOR QUANTIFICATION OF REPLICATIVE LIFESPAN AND OBSERVATION OF SENESCENE

(71) Applicant: CYTOSPAN TECHNOLOGIES CORPORATION, Toronto (CA)

(72) Inventors: Michael James Pettigrew, Toronto (CA); Christopher Douglas Pettigrew, Toronto (CA)

(21) Appl. No.: 14/410,137

(22) PCT Filed: Jun. 20, 2013
(86) PCT No.: PCT/CA2013/050474

§ 371 Date: Dec. 22, 2014

Related U.S. Application Data
(60) Provisional application No. 61/662,137, filed on Jun. 20, 2012.

(51) Int. Cl. C12Q 1/06; G01N 1/34

(52) U.S. Cl. CPC C12Q 1/06; G01N 1/34

(57) ABSTRACT

The present invention teaches an apparatus and method for the automated study of senescence of cells. The apparatus includes a plurality of plates, customized with a flow-through hole, arranged on a platform. A microscope with integrated camera is positioned above the platform and both microscope and platform are coupled to a rotation mechanism to allow relative rotation between the platform and the microscope. Cells are anchored magnetically, chemically, or electrostatically to the plates and are treated to a controlled environment. Daughter cells born from the mother cell are observed by the microscope and then washed, using a wash fluid, through the flow-through hole in the plate. A processor automates the process and allows for a user to input customizable test parameters and value thresholds to indicate a test is completed. The processor also organises test data in the form of a spreadsheet for simple modelling and data manipulation.

### FIELD OF THE INVENTION

The present invention relates to the observations of life forms in conditioned environments.

### BACKGROUND OF THE INVENTION

Replicative lifespan (also called cellular lifespan or abbreviated as RLS) is defined as the number of daughter cells (also called buds) that a mother cell produces before undergoing senescence. The measurement of RLS is a powerful experiment that can be used in many types of research. By knowing the lifespan of the cell in a variety of situations, researchers can determine the effects of various mutations on the cell and use this knowledge to determine biochemical pathways, gene function, and the effects of novel compounds and environmental factors. This information can help determine the causes of various diseases and be used for pharmaceutical development. Replicative lifespan is primarily, but not exclusively, studied in the yeast Saccharomyces cerevisiae, which is a common eukaryotic model organism. The measure of replicative lifespan in yeast is often thought of as a model for human cellular aging.

The current method of determining RLS in yeast requires researchers to use a micromanipulator (a light microscope with attached dissection needle). It involves performing the tedious task of using the small needle to count and manipulate yeast daughter cells. The protocol for the current method is as follows. Yeast cells are placed on a plate and then virgin daughter cells (cells that have never budded before and are therefore at the beginning of their lifespan) are isolated and spread out—these cells become the mother cells that will have their lifespan quantified. The plate is then incubated for approximately two hours, which is about the time it takes for a yeast cell to bud fully. Then, the researcher records the number of buds per mother cell in a spreadsheet, and uses the needle to separate and discard the daughter cells. The plate is then incubated for another two hours, and the process is repeated until all cells reach senescence. When the researcher goes home for the night or weekend, the cells are placed in the refrigerator to slow their cell cycle. It usually takes three or more weeks for 40 wild-type cells—the average number per plate—to reach replicative senescence. The procedure can be inaccurate, with researcher bias, plate degradation, and refrigeration time contributing to random error. Therefore, two or three plates are counted for accuracy. It is a slow and laborious process. This procedure is the rate-limiting step in many forms of research, and can heavily delay the development of drugs.

A process for observing a larger number of cells automatically, without the need for direct human manipulation, would drastically improve the rate of replicative lifespan study as well as strengthen scientific evaluation of these studies. For more information on the current method, see the publication and video by Steffen et al. titled “Measuring Replicative Lifespan in Budding Yeast” (JoVE 2009).

### SUMMARY OF THE INVENTION

An embodiment of an apparatus for observation of senescence comprises:
at least one support plate having a surface for supporting one or more mother cells, said at least one support plate including a flow-through hole for draining liquids,
a wash mechanism including a wash reservoir for holding a wash fluid and a delivery system for delivering the wash fluid to the plate,
a microscope positioned to observe said surface of said support plate, and
a controller programmed for controlling at least the wash mechanism and the microscope.

An embodiment of a method for observing senescence in cells comprises
method for quantifying replicative lifespan and observing senescence in cells, comprising:
preparing at least one plate,
immobilizing at least one mother cell capable of producing an offspring cell to the plate,
observing production of offspring cells by the mother cell with a microscope,
washing the plate with a wash fluid from a wash reservoir such that offspring cells are removed from proximity of the mother cell, and
determining the replicative lifespan of the mother cell by observing the total number of offspring cells produced by the mother cell.

A further understanding of the functional and advantageous aspects of the invention can be realized by reference to the following detailed description and accompanying drawings.

### BRIEF DESCRIPTION OF THE DRAWINGS

The invention will be more fully understood from the following detailed description thereof taken in connection with the accompanying drawings, which form part of this application, and in which:

FIG. 1 shows a front and side view of the collective primary embodiment of the invention. It displays the wash reservoir, the computer, the microscope, a cover, a wash liquid sink, the cell-containing plates, a rotating platform, a light source and a base.

FIG. 2 shows the isolation of a virgin daughter cell in the set-up process from an anchored mother cell.

FIG. 3 shows a top view of a portion of the rotating platform displaying two plates. One plate shows five cells subject to a flow of wash fluid. Said flow is dragging the yeast daughter cells through the drain hole following the arrows.

FIG. 4 shows a concept sketch of a possible embodiment of the invention. This design uses the concept of a removable “cartridge” or “magazine” which can be loaded with the prepared plates. The cartridge indexes in a vertical motion to allow access to each of the plates for inspection and/or cell removal. The base would need to be heavily weighted for ballast so that the device is not front heavy. The touchscreen or display is presented forward towards the operator to optimal visibility and access. The challenges with this concept would include maintaining the incubation temperature within the cartridge and fitting the actuation mechanisms within the thin side profile.

FIG. 5 shows a concept sketch of a possible embodiment of the invention. This concept was derived from an automation system which uses adapter plates on a conveyor belt. The adapters would be part of the conveyor belt and would allow the plates to be locked into them. This would allow the plates to travel around the conveyor where they would be inverted and upright at various positions. The idea behind this layout was that the majority of the system would be held at the incubation temperature, with the Vision Inspection (VI) module (the imaging system) located at the top, looking down onto the plates as they pass through the field of view. The wash/cell removal process could be separated from the VI system by occurring underneath the conveyor allowing easy removal of waste fluid and separation of the wet and dry areas of the device. The sketch does not illustrate the fact that there would need to be a front cover on the device which would complete the enclosure for the incubation chamber.

FIG. 6 shows a concept sketch of a possible embodiment of the invention. This design is a concept derived from a compact disc change mechanism. This mechanism could potentially be oriented horizontally as shown here, or in a vertical orientation. This is one of the more space efficient designs with the plates stacked one on top of the other. A front facing interface screen gives good visibility and screen access to the user.

FIG. 7 shows a concept sketch of a possible embodiment of the invention. This layout was conceived from an airport luggage carousel. It keeps the plates oriented in the same direction in all locations by mounting them to a flat moving conveyor. This design has a smaller footprint than a circular carousel. The layout would be such that the length of the device would be oriented front to back on the bench so that the minimum width is taken up.

FIG. 8 shows a concept sketch of a possible embodiment of the invention. This concept was derived from the layout of old slide carousels. This layout creates a very efficient footprint, and also creates the opportunity to isolate the wet area from the electronics and controls. The dome containing the plates would be the incubation chamber. The slides would rotate on the carousel in the incubation dome, and then one plate at a time would be ejected forward towards the VI module. In this case the washing is shown to happen here as well, but an alternative could be to have the washing occur within the dome at the back.

FIG. 9 shows a concept sketch of a possible embodiment of the invention. This is a standard rotating carousel design. This is a simpler mechanism than some of the previous design suggestions but its major drawback is the large footprint required to accommodate the ten plates. This option may be more cost effective than some of the other suggested layouts.

FIG. 10 shows a concept sketch of a possible embodiment of the invention. This concept came about from the idea of trading off the development and hardware costs of the mechanisms required to move the plates around with the cost of ten cameras. The plates are placed on one of two drawers which are manually opened and closed. Once in the device the plate does not move. Each plate has its own dedicated camera. The viability of this design would depend on the hardware cost and the performance of the cameras chosen. This concept also allows some more flexibility with how the cells are viewed as the researcher would be able to constantly monitor the cells growth rather than just viewing it in intervals.

FIG. 11 shows a concept sketch of a possible embodiment of the invention. This concept uses two cameras moving in a single linear axis over the top of a 2×5 array of plates. This approach simplifies the motion requirement, and reduces the footprint, with the trade-off being that two cameras are required. The plates, once manually loaded into the device would remain static throughout the course of the experiment.

FIG. 12 shows a concept sketch of a possible embodiment of the invention. This design concept was created to explore a way to create a space efficient, floor standing device. The plates are static in this design, mounted vertically one above the other. All the movement is performed by the optical assembly with moves on a macro level vertically, but also has micro movements in the x-y plane over the plates. In this layout, the entire vertical tower would be held at the incubation temperature. If required for cell removal, a wash and drain system would need to be incorporated into the tower with waste fluid at the bottom of the tower.

FIG. 13 shows the coupling of the mother cell (left) and the progenitor cell (right) due to the application of the nocodazole. A daughter cell is seen budding from the mother cell.

FIG. 14 shows a diagram of the device architecture (i.e. what the processor controls).

FIG. 15 shows a flowchart of a possible sequence of events for the device to determine the replicative lifespan of 60 cells per plate for 10 plates total.

### DETAILED DESCRIPTION OF THE INVENTION

Generally speaking, the systems described herein are directed to an apparatus and method for observation and quantification of cellular replicative lifespan, defined as how many times a cell buds or divides until senescence is reached. As required, embodiments of the present invention are disclosed herein. However, the disclosed embodiments are merely exemplary, and it should be understood that the invention may be embodied in many various and alternative forms.

The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to an apparatus and method for quantification of cellular lifespan.

Without limitation, the majority of the systems described herein are exemplary embodiments. It should be understood that the invention may be embodied in many various and alternative forms and that the following should not limit the scope of the invention.

An offspring cell, also called a daughter cell or a bud, is defined as any cell produced by another cell by division, fission, budding, or any other process.

The word plate can refer to a Petri dish, a slide, a test tube, or any other device that can hold one or more cells. The plate may or may not be enclosed, and may or may not contain solid or liquid growth media.

A controller is a device that controls the function of various system components. Examples of controllers are processors, microprocessors, microcontrollers, and computers. Software is used on these devices to instruct the system components controlled by the controller on their function and timing.

The present disclosure discloses a new innovative apparatus and method for measuring the replicative lifespan of cells. The introduction of a system to observe the whole lifespan (from birth to senescence) of a large number of cells in a controlled environment in a rapid and automated fashion significantly improves upon the traditional system of direct human manipulation and observation, a method that is unreliable, slow, and very laborious. This novel system uniquely combines sophisticated image processing and automation software, hardware, and biochemistry in a bench-top laboratory instrument to automatically observe single cells and determine their replicative lifespan. This measurement of replicative lifespan applies to all types of cells, including but not limited to yeast cells, stem cells, cancer stem cells and neuroblasts.

It is advantageous to be able to test large samples of cells concurrently to more quickly and accurately measure the effects of chemical environments, stimuli, and genetic mutations on said cells (rather than counting two or three plates with 40 cells each using the current method). As the cells are exposed to the same environment, this will be attributed to a higher level of confidence in the scientific study. Likewise, an automated solution would allow different researchers to compare results with one another. Previously, this was impossible as each person would typically get very different results performing the same experiment, as they had different “counting styles”. An embodiment of the present invention allows a much larger sample of cells to be tested per plate, and allows many plates to have different conditions (such as drugs mixed in with the solid growth media present) and to be tested at one time. Furthermore, it is also far faster than the current method, as it can be set to work 24 hours a day. Subsequently, it removes the need to transfer cells to the refrigerator—a large source of error in the current experiments.

The device functionality is briefly summarized as follows: The user prepares the cells on plates (slides can also be used). This is done by isolating virgin daughter cells (cells that have never budded before)—these will become the mother cells to be studied. These mother cells are then immobilized in such a way that they remain anchored for the entire experiment, while daughter cells that bud continuously from them are not anchored. The plates or slides are then inserted into the device, and the device begins its operation. The cells are maintained at a constant temperature to promote replication. At specific time intervals (generally two hours for yeast), an image is taken of the cells and the number of daughter cells each mother cell has produced is counted. The daughter cells are then washed away by the wash mechanism, leaving only the mother cells behind. Another image is then taken to confirm the daughter cell removal. The process of incubation, imaging, and washing is then repeated, increasing the count each time by the number of daughter cells that have been removed. The replicative lifespan of each cell can then be determined as the total number of buds that each mother cell produces. An average replicative lifespan is often produced to increase accuracy, as there are many cells studied in each experiment.

A feature disclosed herein in a specific set-up process using the drug nocodazole. Applying biotin (a compound that binds streptavidin or avidin) to cultured cells, and then applying nocodazole, will allow the creation of a non-biotinylated cell (the mother cell to be studied) attached to a progenitor cell (“support structure”) which is biotinylated. This progenitor cell can be attached to streptavidin or avidin directly on a plate, or to streptavidin-coated magnetic beads, which are then held in place by magnets. This allows wash fluid to wash daughter cells away while the mother cell stays immobilized. It also allows the user to be certain that all mother cells to be studied are virgin daughter cells, as all cells that are attached to progenitor cells after the application of nocodazole have never budded before. Finally, it allows easy recognition and continued tracking of each mother cell.

Another feature disclosed herein is the imaging system. The imaging system is the combination of imaging hardware generally including a microscope (or a lens capable of magnification), a camera, and imaging software. Different types of imaging including but not limited to bright field, dark field, phase contrast, differential interference contrast, confocal, and fluorescence imaging can be used. The uses of the imaging software include keeping track of individual mother cells and counting the daughter cells. Not only will this device provide accurate measurements of a cell's lifespan, it can provide continuous imaging (if only one plate or slide is being studied) or to images in interval (if multiple plates are being studied) of a cell's entire lifespan. Researchers can combine information on the lifespan of a cell with images of the cell, and thus study its morphology during aging. If the device possesses the capabilities of its own fluorescence microscope, or an external one if the device is built in a modular approach, researchers can combine information on the lifespan of a cell with information on the localization of fluorescently-labeled proteins or other molecules. For instance, if a researcher sees a sudden drop in viability in a lifespan curve they can compare this to the activity of a certain protein or molecule at that precise time and determine its role in determining lifespan.

The image processing subsystem is responsible for a number of critical steps in the determination of RLS. It assists with finding the correct location of the objective and field of view over the cells of interest, it identifies the mother cell, it identifies the newborn daughter cells and differentiates between the mothers and daughters, it counts the newly replicated daughter cells, and it assists with confirming the removal of the daughter cells after counting. If a daughter cell fails to be removed, it will recognize this and make sure that the daughter cell is not counted multiple times.

Another feature disclosed herein is the wash mechanism. This is designed to wash only the daughter cells away, when combined with the immobilization outlined above. The wash mechanism has three different aspects: the flow-through capabilities of the custom plates or slides, the specific wash protocol, and the immobilization of the cells to the custom plates. The custom plates disclosed herein have flow-through capabilities. Each plate is customized to have a flow-through hole oriented towards a sink. The device utilizes a wash mechanism that passes wash fluid through said holes in said plates at specified time points. This allows the daughter cells to be carried away from the mother cell upon cell division. This also allows the user to control the flow of fluid that the cells are exposed to and better replicates the real condition such a cell would encounter in the researcher's desired environment.

The moving platform disclosed herein allows for concurrent testing of multiple different plates or slides, which could each have different test environments and conditions, further providing control abilities to the user as well as the ability to test a large number of cells at a time. Previously, the number of cells studied per plate, and number of plates studied at one time, was limited by the number of technicians available and their workload capabilities. To simplify the explanation of this aspect of the invention, we refer to a rotating platform. However, this is only one possible embodiment. The purpose of the movement mechanism on the platform that contains the plates is to pass each of said plates under the microscope and wash mechanism in turn.

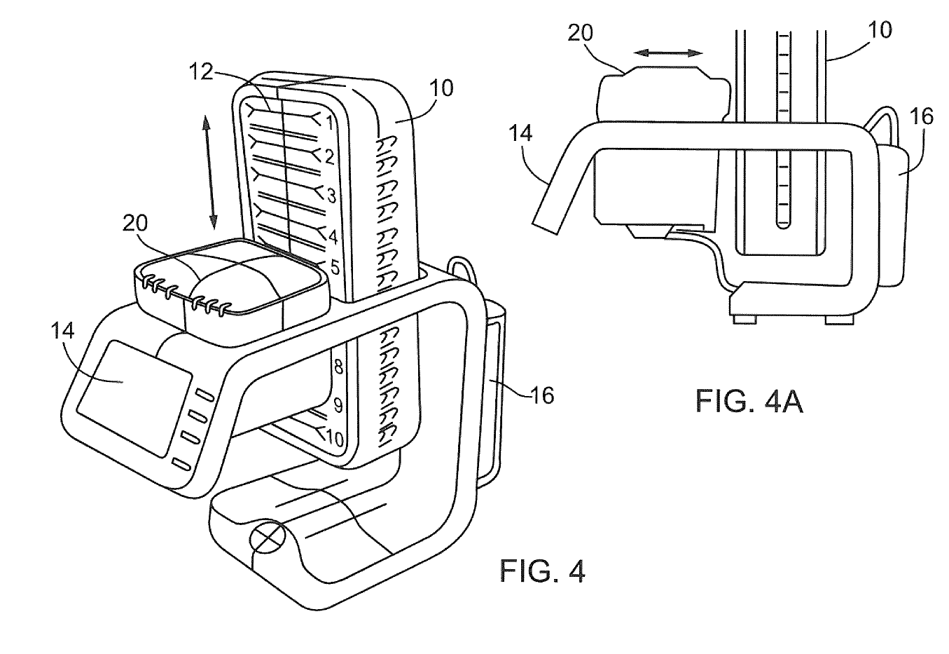

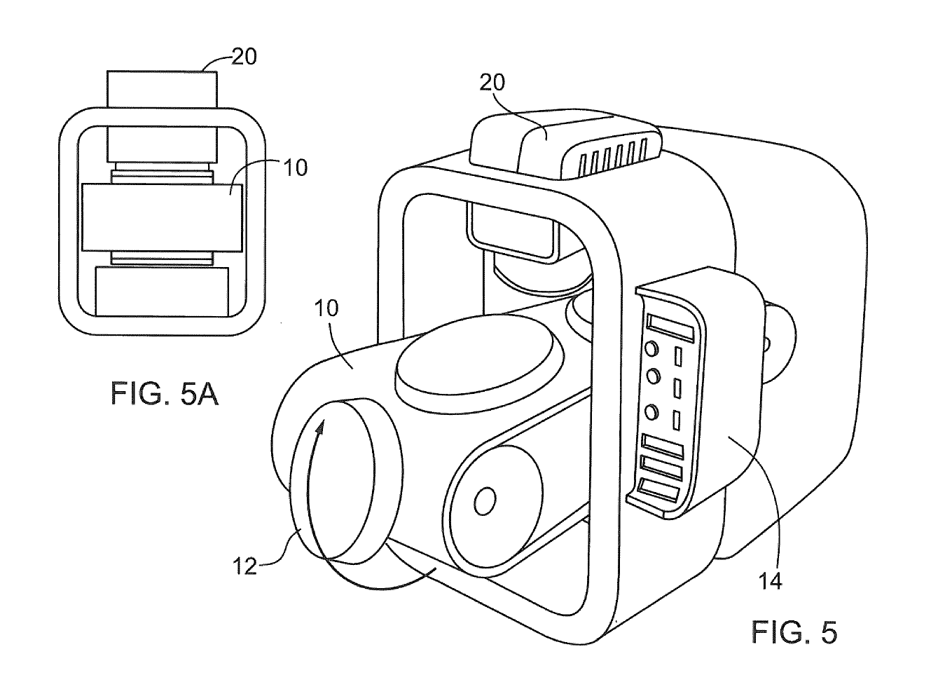

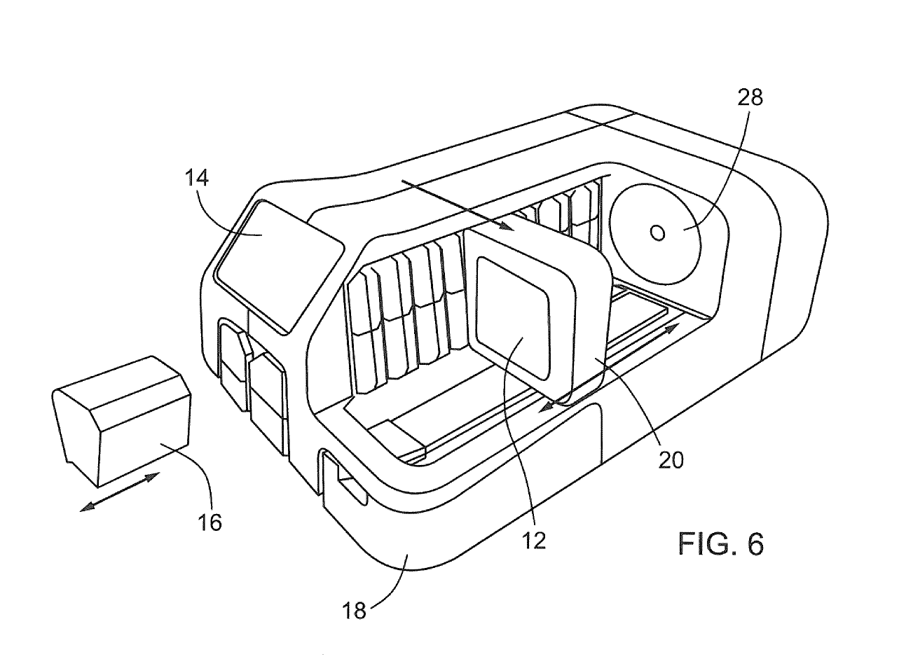

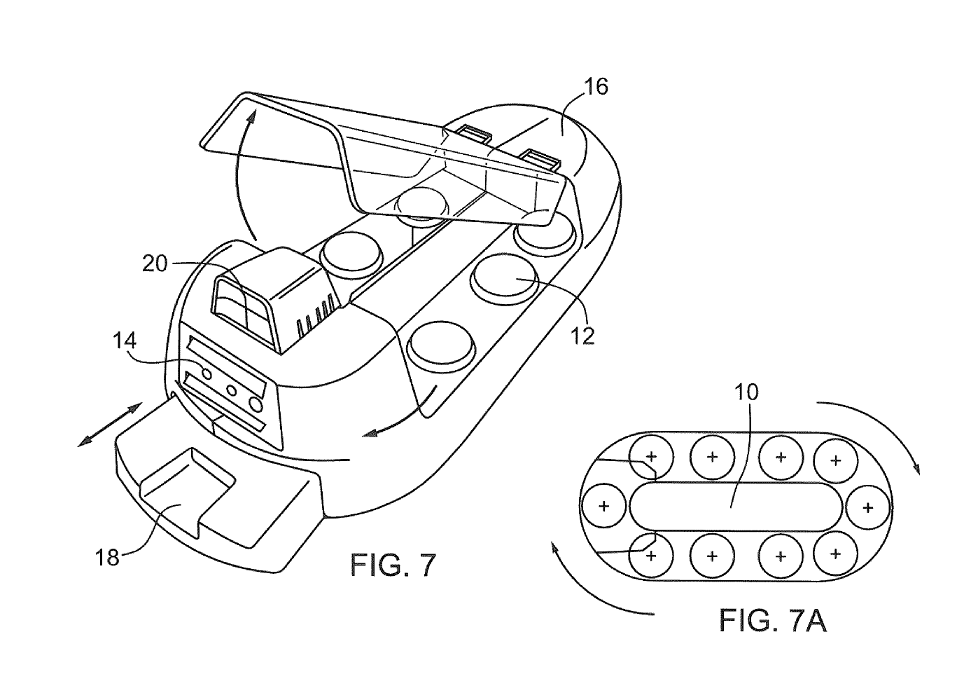

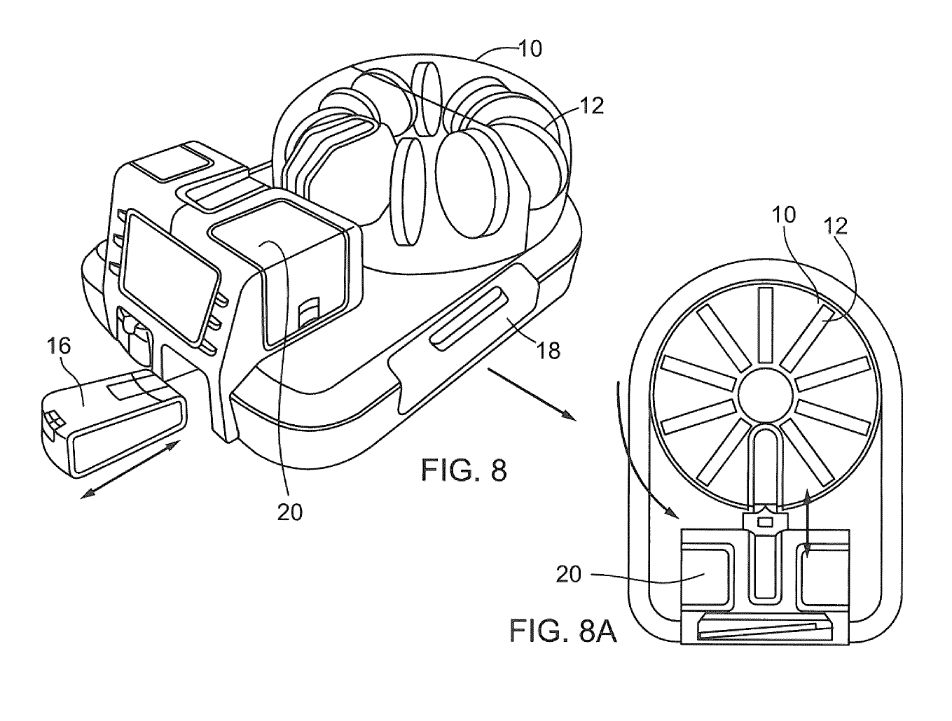

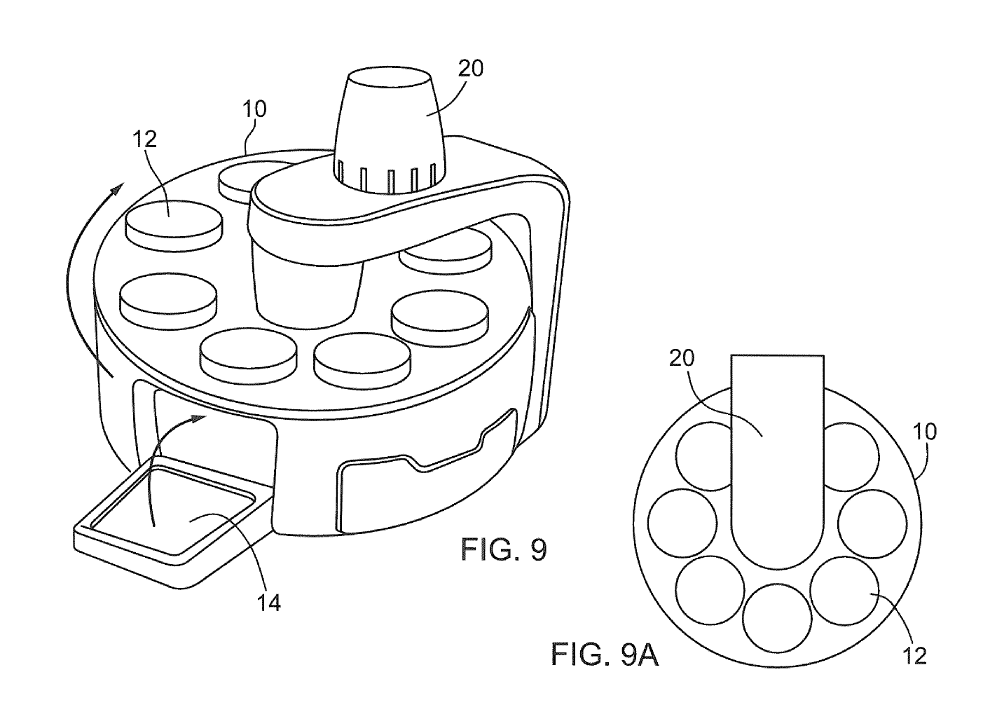

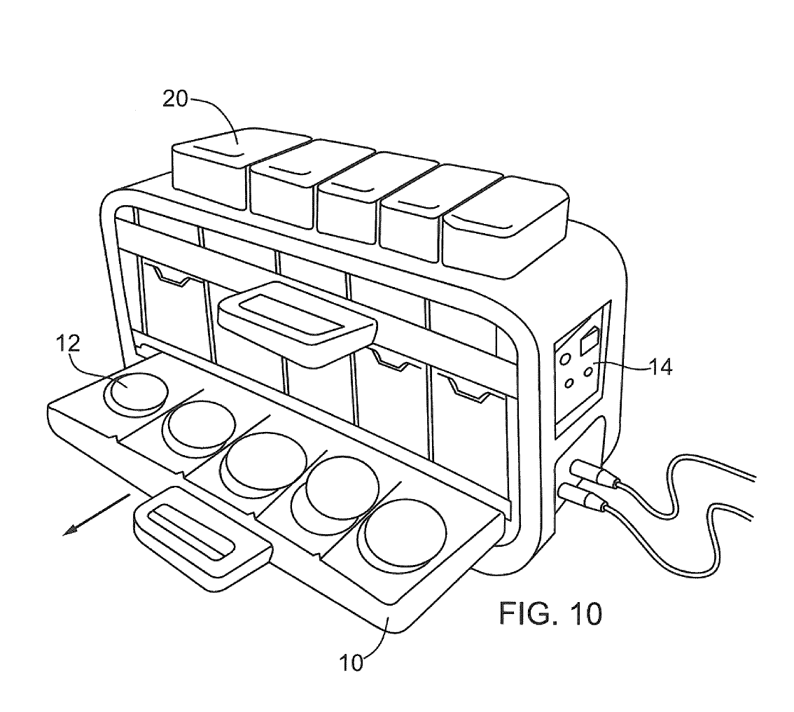

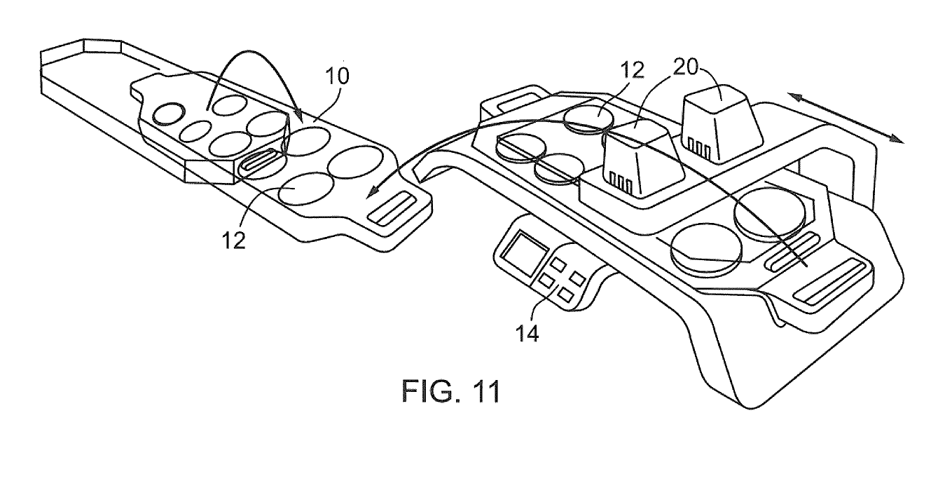

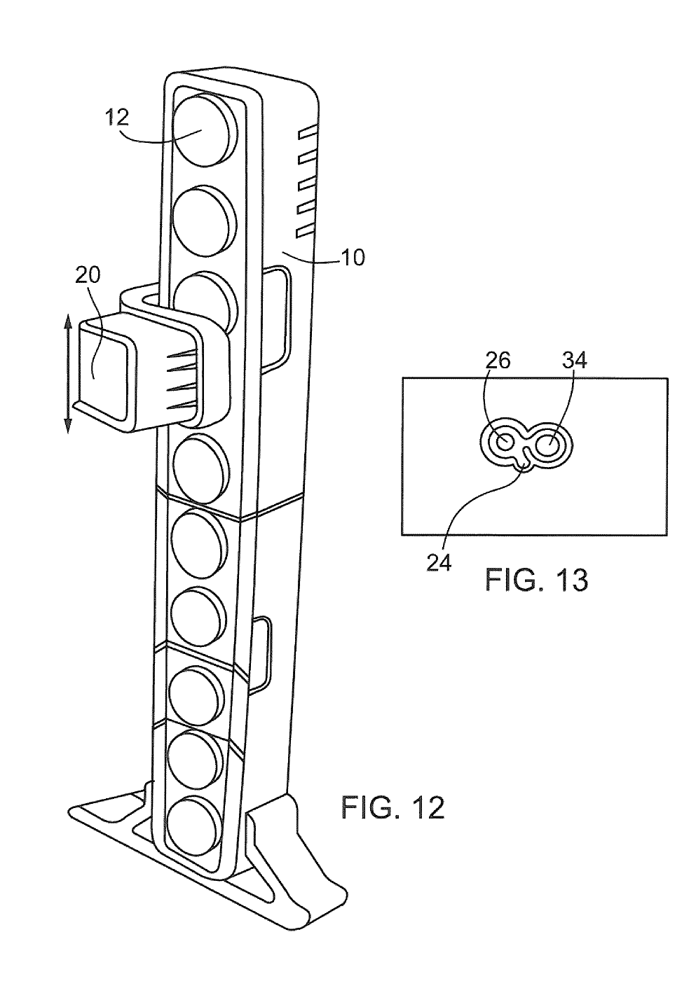

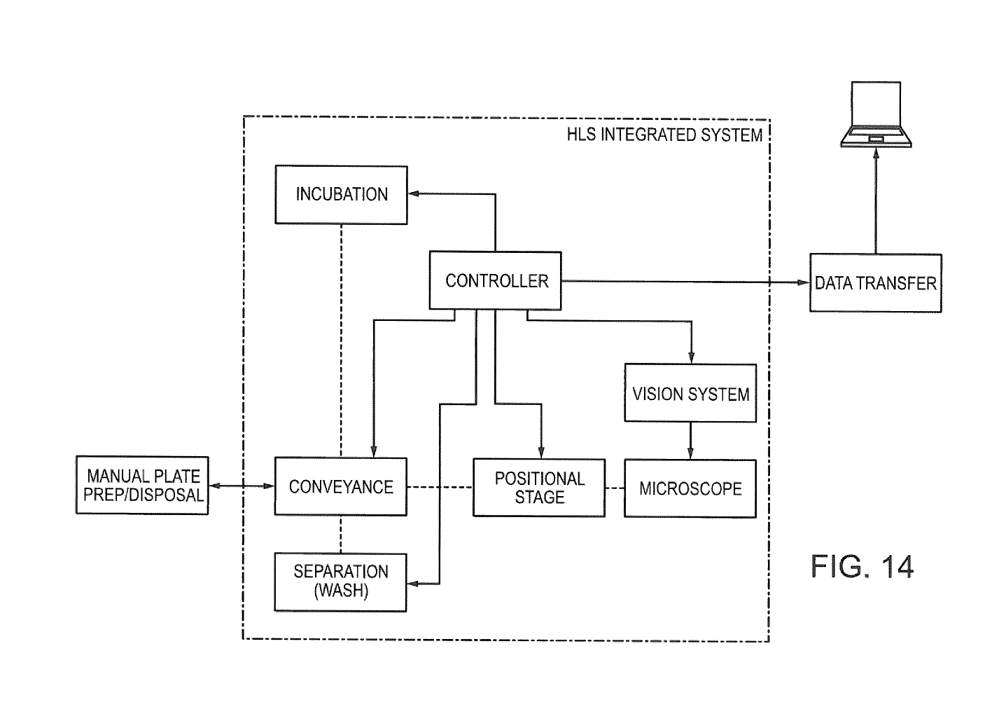

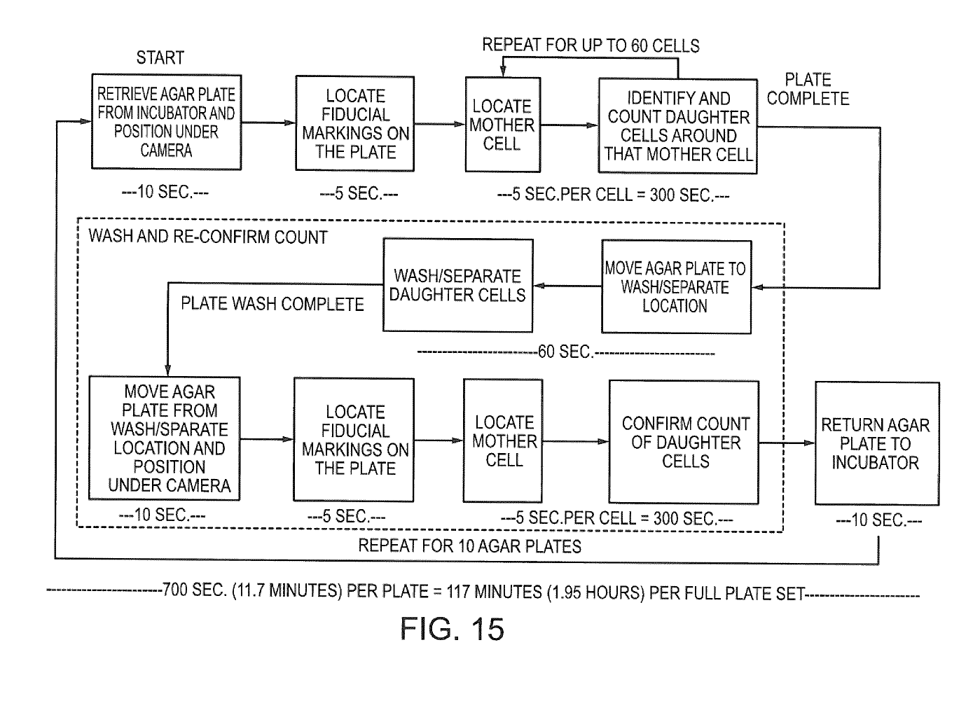

The device is envisioned as a bench-top instrument for use in a typical university or industry laboratory. However, larger versions capable of determining the replicative lifespan of more cells, or larger organisms, may have their base on the floor, or be present in separate rooms. Drawings of embodiment of the present apparatus are shown in FIGS. 4 through 12, although the design is not limited to these concepts.

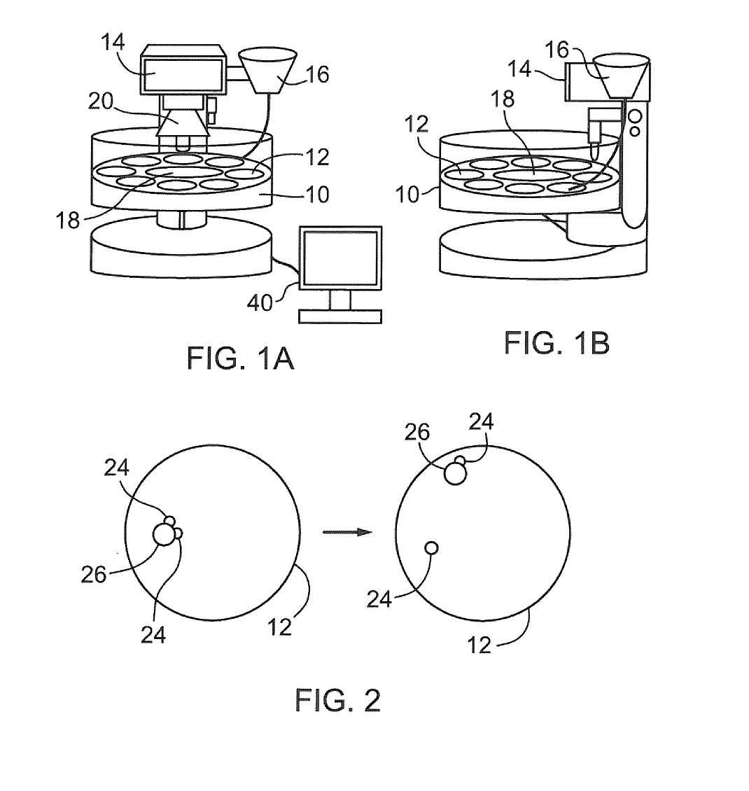

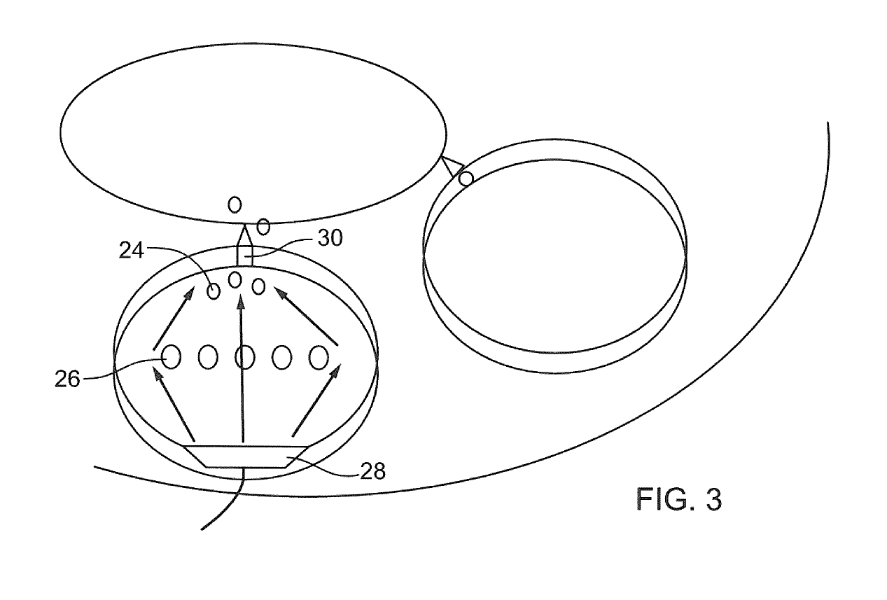

One embodiment of the present invention is used to measure the replicative lifespan of Saccharomyces cerevisiae yeast cells. The device can, however, also measure the lifespan of other types of cells including but not limited to stem cells, cancer stem cells and neuroblasts. These cell types often divide asymmetrically in the same fashion as yeast cells. Unlike yeast cells, however, stem cells can also divide symmetrically, essentially forming a second mother cell 26. Software for devices for these types of cells will be able to track both newly created mother cells 26 if symmetrical division is present. This embodiment is depicted by FIG. 1, which consists of a rotating platform 10 which can be heated to various temperatures, such as 30° Celsius for yeast experiments. The heating of this platform 10 essentially makes it an incubator, maintaining cells in their optimal growth temperature. This platform 10 will hold Petri dishes (also called plates) 12, modified with a flow-through hole 30, each 10 cm in diameter. The platform 10 will be covered to maintain the temperature and to prevent contamination of the plates 12. Alternatively, a possible embodiment of the present invention would utilize test tubes or slides rather than plates 12. Another embodiment will not rotate or move, and will have a single plate 12 beneath a microscope 20.

The embodiment further comprises a wash reservoir 16 and wash mechanism 28 to dispense a fluid (liquid or gas) onto each plate 12 at each time point, or continuously. This fluid will then drain into the wash fluid disposal 18 in the center of the platform 10 through a hole 30 in the side of the specialty plates 12. FIG. 3 demonstrates this set-up, with the wash mechanism 28 positioned at the opposing end to the flow-through hole 30 in the plate 12. Wash liquid is then dispensed at a rate fast enough to carry away unwanted daughter cells 24 through the hole but not so fast as to displace the mother cells 26 being studied. Although the platform 10 will be covered, this is not shown in the diagram for simplicity. The liquid used will depend on the cells. A variation of this is to use multiple wash fluids and reservoirs 16 where one wash fluid is used to remove buds 24, while the other is used for the application of drugs or compounds in liquid form. Another variation is the use of gas as opposed to liquid as the wash fluid. Chambers or reservoirs 16 that can contain washing fluid may also be located on each individual plate 12 or slide.

The embodiment further comprises a touch screen computer 14, which displays information and allows the user to enter relevant information needed for device operation. FIG. 1 shows an embedded computer 14 at the top of the machine, but another option is to place it on the base or on the side. This decision will be based on user comfort. An external computer 40 may instead be used.

The microprocessor 14 (or 40) is equipped with software to synchronize the activity of the wash reservoir 16 pump, the orientation of the rotating platform 10, the microscope 20 and the storage of images. The software is configured to control the automation of the entire device. It is programmed to perform a photograph then wash operation at each time interval for each plate 12. The software will analyze these photographs at each time interval, and determine the number of buds 24 per mother cell 26. It will then analyze another photograph to ensure all buds 24 have been removed (in order to prevent buds 24 from being counted multiple times). The software is also equipped with the ability to initially identify a mother cell 26 on the first iteration and subsequently recognize said same mother cell 26 on further iterations of the experimental cycle.

Finally, the imaging system 20 used in the present embodiment may be a microscope 20 with an integrated camera 20. The magnification used will be one where mother cells 26 and daughter cells 24 can be differentiated based on size. While the primary embodiment utilizes a light microscope with bright field, dark field, phase contrast, and/or differential interference contrast, a variation on this is the use of a fluorescence or confocal microscope that can track the location of a fluorescently-labeled molecule as the cell ages. The optical subsystem component of the imaging system 20 consists of five main components: 1) the illumination source which can be a tungsten halogen lamp or LED/laser source, 2) a condenser stage, 3) the plate 12 or slide in the imaging plane, 4) the objective stage, 5) the detector stage which is a CMOS or CCD detector. An alternative embodiment of the apparatus comprises the microscope 20 and platform 10 being configured such that the microscope 20 rotates about a fixed platform 10.

The current embodiment of the present invention is intended to be a stand-alone device with an on board computer 14 or 40 and human machine interface. It is intended to reside on a standard laboratory bench with other standard laboratory equipment.

Another embodiment does not include a microscope 20, and fits on the stage of an existing fluorescence microscope 20 in a modular format. It uses the capabilities of the existing microscope 20 (a fluorescent microscope in one embodiment) to image the cells and track fluorescently-labeled molecules inside the cells. It controls the microscope 20 through external software, which synchronizes it with the rest of the device including the wash mechanism 28, incubator and rotation platform 10.

To begin the process of using the invention to determine the replicative lifespan of cells, the plates 12 or slides must first be prepared. This means that 1) virgin daughter cells (cells that have never budded before and are therefore at the very beginning of their lifespan) must be isolated—these will become the mother cells 26 to be studied, and 2) these mother cells 26 must be immobilized to the plate 12 or slide in such a way that they remain immobilized for their entire lifespan while the daughter cells 24 that are budded off of them are not immobilized. A number of methods to immobilize the cells are possible, and include immobilization through biochemical means or physical entrapment. It should be understood that the below specific embodiments are examples of many possible methods for immobilizing and lifespan measurement of cells on a plate 12 and that they should not limit the present invention.

A primary embodiment of the invention uses the following set-up method. It makes use of a “support structure” created on each mother cell 26 being studied. These support structures can be treated with immobilizing drugs, allowing researchers to keep track of the same cells (the mother cells 26) for the entire experiment while not applying immobilizing drugs to these mother cells 26 (potentially altering the RLS and changing the data). A specific example of a support structure being created is based around the use of a microtubule-inhibiting compound called nocodazole. This drug arrests the cells in their G2/M stage of cell division, so that each cell becomes a dumbbell-shaped grouping of two cells. This can be seen in FIG. 13. One of the cells in the dumbbell complex is the progenitor cell 34, the cell to which nocodazole was originally applied, while the other cell is the mother cell 26. This progenitor cell 34 is the support structure, which can be treated with immobilizing compounds. The replicative lifespan of the mother cells 26 will be determined. The two cells in the dumbbell are completely separate, as there is a septum between them. The progenitor cell 34 simply acts as the support structure, mentioned above, for the mother cell 26. Prior to applying the nocodazole, biotin is applied to the progenitor cell 34. This biotin is able to bind to streptavidin or avidin, one of which is bound to the plate 12 or slide. Streptavidin and biotin have a high affinity for each other, and procedures based around this property of these compounds are used in many aspects of biotechnology.

The end product of these biochemical steps is the dumbbell-shaped pair of cells, with an actively budding mother cell 26, where only the progenitor cell 34 is immobilized on a plate 12. In this embodiment, there is no biotin directly on the mother cell 26. As the mother cell 26 buds, the daughter cells 24 produced are continuously counted and washed away. This specific embodiment allows a researcher to start with a random population of cells, thus making the set-up very easy, but at the same time makes sure that all cells being analyzed are virgin daughter cells (meaning they have never budded before and are therefore being studied from the start of their lifespan). The mother cells 26 begin their lives as virgin daughter cells of the progenitor cells 34, as all the cells in the culture are synchronized from the nocodazole cell cycle arrest.

The following laboratory protocol was used by the inventors. First, the researcher starts an overnight culture from yeast cells to an optical density of 0.5. The next morning, once the cells have re-entered the log phase, the cells are resuspended in phosphate-buffered saline and biotinylated with 3.5 mg biotin/0.67 mL. The cells are vortexed, spun down, washed then resuspended in growth media. Next, 15 ug/mL nocodazole is added to the cell culture. This nocodazole is often mixed with 1% dimethyl sulfoxide. The cells are allowed to grow in this nocodazole for three hours at 30° C. The nocodazole halts the yeast cell division cycle at the G2/M phase, causing each cell to take on the morphology of a dumbbell-shaped grouping of two cells. The cells are then spun down, washed and resuspended multiple times to remove the nocodazole. The cells exit the nocodazole cell cycle block and resume budding. The cells are then sonicated, and streptavidin-coated paramagnetic beads are added. The cells are then placed on ice for two hours, and a magnet is used to isolate the biotin-labeled cells. It should be noted that various aspects of this experimental protocol may be altered with the same effects, and this description is an example of one of the possible protocols.

The cells are deposited on the plate 12, which has a magnet to isolate the cells and hold them in place. Now, the mother cells 26 that are being studied are attached to progenitor cells 34 which are biotinylated and are attached to magnetic streptavidin beads which are held in place by magnets.

An alternative to this set-up process does not use magnetic beads or magnets. The plates 12 may have patterns of streptavidin or avidin streaked directly on them to hold biotinylated cells in place.

Another biochemical method of immobilization does not use nocodazole, and instead only uses biotin on the mother cells 26. This biotin is then bound to streptavidin or avidin on the surface of custom plates 12, or onto magnetic streptavidin-coated beads as in the previous method, and are then held in place by magnets.

Other examples of immobilization using biochemistry are included here. Common methods of immobilization are aggregation to other chemicals, absorption into other materials, or entrapment in fluids. The method of aggregation of a cell onto a plate 12 is described by Lantero, Jr. in “Immobilization of enzymes.” (U.S. Pat. No. 4,760,024). Lantero teaches a method of reacting the membrane of an enzyme or a microorganism with polyethylenimine and further adding a chitosan and glutaraldehyde solution. The reaction produces a cross-linked chemical compound that can be removed from the solution and anchored to a plate 12.

The method of absorption into other materials is described by Zuffi et al. in a “Process for immobilizing cells on a resin” (US2007/0249023). Zuffi teaches a process which includes a step of a resin absorbing a cell, essentially immobilizing it in the resin.

The method of suspension or entrapment of a cell in a viscous fluid is described by Chen et al. in “Immobilization of microorganisms or enzymes in polyvinyl alcohol beads.” (U.S. Pat. No. 5,290,693) Chen teaches a method of combining polyvinyl alcohol solution with the cells to form viscous beads. The cell is contained within the bead and is thereby immobilized.

Specifically, in another embodiment, after virgin daughter cells (which will become the mother cells 26 to be studied) are isolated and separated on a plate 12 or slide, they are coated with 0.2% polyethylenimine (PEI), and adhered to a material with a surface-negative charge such as glass or sand which can be mixed into the growth medium. This binding is not affected by a stream of running water, making it quite strong, and the viability and growth patterns of cells coated with PEI are not significantly different from those that were not.

In addition, physical immobilization can be used. This method generally consists of a system that physically holds or traps the mother cell 26 in place. Due to the size difference between the mother cell 26 and the budded daughter cells 24, the daughter cells 24 are not held in place, and are instead washed away. This immobilization method is described by Lee et al. in “Whole lifespan microscopic observation of budding yeast aging through a microfluidic dissection platform” (PNAS 2012), and Zhang et al. in “Single cell analysis of yeast replicative aging using a new generation of microfluidic device” (PLOS One 2012).

There are multiple possible procedures to deposit the cells on the plates 12 for the nocodazole embodiment of the device. The first uses the micromanipulator to lay all the mother cell 26—progenitor cell 34 complexes in a grid. This ensures that all cells will be far enough away from other cells to be analyzed by the imaging software. Another procedure lays the cells on the plate 12 randomly. Only cells far enough away from other cells to be accurately analyzed by the imaging software will be analyzed for the experiment. The position of magnets, or of streptavidin or avidin located directly on the plate 12 or slide, can also be used to control how the cells lay on the plate 12 (for example, place them all in a row).

For embodiments that make use of other methods of immobilization, the micromanipulator will be used in the set-up process to isolate virgin daughter cells (which will become the mother cells 26 being studied) and to spread them out on the slide or plate 12.

In order to insert the prepared plates 12 into the instrument the user must open the cover and place the pre-prepared plates 12 into one of the plate holders on the rotating platform 10 of the device. The platform 10 will have a marking, which tells the user where the hole in the plate 12 should fit, and the plate 12 will fit to face in that direction. This will allow the wash mechanism 28 to perform as intended and reduce error.

The following information will be entered into the computer processor 14 or 40 at the discretion of the user. Information about the strain (such as mutations and the strain background) and plate 12 conditions (such as glucose percentage and presence and concentration of drugs), the location of each mother cell 26, the focus level for the area on the plate 12 where the mother cells 26 are, will be entered in an embodiment where software and detectors are not in place to provide the computer 14 or 40 with such data already. There may be multiple focus levels if the plate 12 is not poured level.

Once the automated replicative lifespan routine has been started there is no need for the operator to remain at the instrument—no interaction is required. The operator is free to perform other laboratory tests while the automated replicative lifespan measurement is underway.

The protocol for the device in the current embodiment heats the environment to 30° Celsius (if the device is not already in use), and incubates the cells for two hours.

The device will then take a picture and the image processing software will count the number of buds 24 for each mother cell 26 (usually a number from 0 to 3). It may change the focus of the microscope 20 to a predetermined level for each plate 12 to accomplish this. It will record the location and identity of each mother cell 26, and store this knowledge between time points, when the plate 12 is not under the microscope 20 (i.e. be able to find the same mother cell 26 every time point).

After the number of buds 24 has been identified, the wash mechanism 28 activates. The specific wash protocol depends on the immobilization technique used earlier. For the nocodazole primary embodiment the protocol can be as follows. If the researcher has chosen to growth the yeast on solid media, a wash mechanism 28 will pass wash fluid over the plate 12, washing away the daughter cells 24 through a hole in specialty plates 12 and down a funnel 18 into the discarded wash fluid in the centre of the platform 10. In another embodiment, the fluid only moves the daughter cells 24 far enough away to prevent development of a colony.

If the researcher has chosen to grow the yeast in liquid media, the old liquid media will be drained (taking the daughter cells 24 with it) and new liquid media will be pumped in to refresh the yeast cells. The mother cells 26 will not be washed away, as they are immobilized in the set-up process. Another possibility is the use of a constant stream of liquid growth media.

Another photograph will then be taken to ensure all buds 24 have been removed and to make sure that any daughter cells 24 that were counted at the previous time point but failed to be washed away are not counted again. In other words, it will take a picture, perform a wash, and then take another picture. The difference in the number of buds 24 between the two pictures will be counted and stored.

The next plate 12 will then undergo this whole process. This process will be repeated every two hours. In other words, the cited embodiment will run a cycle of photograph-then-wash, or photograph-then-wash-then-photograph, automatically every time point.

The following control software protocol is suggested by the inventor but should not limit the many alternative compatible protocols. In this particular embodiment the device control software will incorporate the following functions.

The time between time points can be customized. For instance, a time point can occur every hour or half hour, or other time interval, to decrease the risk of the mother cell 26 being lost among its buds 24 and increase experimental accuracy.

The process will stop once all mother cells 26 have reached senescence. The protocol will be able to detect when a cell or series of cells has reached senescence (i.e. when there have been zero buds 24 for multiple time points) and be able to view progress of the content of each plate 12 before it is finished. The software can halt function when it has determined that all cells have reached senescence. The parameter in testing for senescence is generally the number of time segments the cell undergoes without budding. In the preferred embodiment, this is set to a default of three time points.

The described embodiment will provide the user with the ability to, at any point in the process, view images of the cells or to check if the results to that point are as expected.

The device will then use the data for each individual mother cell 26 to calculate overall replicative lifespan curves, which involves averaging the lifespan of all cells in an experiment. It will perform statistical analysis and comparisons (including the Mann-Whitney U test, also called the Wilcoxon rank-sum test) for each data series. As well as outputting the lifespan of each cell, the device will calculate the amount of time from birth to death for each cell, and the amount of time spent in a post-mitotic stage after replication ceases. Also, continuous or near-continuous images of tracked cells from birth to death will be outputted, as well as activity of fluorescently-labelled molecules if the embodiment has fluorescence capabilities.

The current embodiment will then save multiple data series and be able to export these to an external computer 14 or 40 in a spreadsheet (Excel-compatible) file giving the subject the option to model the data. The processor 14 or 40 will also provide the user with the option to customize the amount of time between time points (i.e. how often the camera 20 takes a picture and washes away the buds 24).

As used herein, the terms “comprises”, “comprising”, “including” and “includes” are to be construed as being inclusive and open-ended. Specifically, when used in this document, the terms “comprises”, “comprising”, “including”, “includes” and variations thereof, mean the specified features, steps or components are included in the described invention. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

1. An apparatus, comprising:
at least one support plate having a surface for supporting one or more mother cells, said at least one support plate including a flow-through hole for draining liquids,
a wash mechanism including a wash reservoir for holding a wash fluid and a delivery system for delivering the wash fluid to the plate,
a microscope positioned to observe said surface of said support plate, and
a controller programmed for controlling at least the wash mechanism and the microscope.

2. The apparatus according to claim 1 wherein the one or more mother cells are selected on the basis of being capable of producing at least one offspring cell.

3. The apparatus according to claim 2 including an immobilization means for anchoring the one or more mother cells to the plate using an immobilizing means for immobilization.

4. The apparatus according to claim 3 wherein the at least one offspring cell is not immobilized.

5. The apparatus according to claim 3 wherein the immobilizing means includes nocodazole applied to a progenitor cell resulting in a cell cycle block which produces the mother cell such that the mother cell is anchored to the progenitor cell.

6. The apparatus according to claim 5 including means for immobilizing said progenitor cell comprising a biotin coating on said progenitor cell and a plurality of avidin- or streptavidin-coated magnetic beads adhered to the coating of biotin, and including a magnet adhered to the plate.

7. The apparatus according to claim 5 including means for immobilizing said progenitor cell including using any one or combination of biotin, avidin or streptavidin to adhere the progenitor cell to the plate.

8. The apparatus according to claim 5 including means for immobilizing said progenitor cell including coating the progenitor cell with a polyethylenimine coating and treating the plate with a surface-negative charged substance wherein said polyethylenimine coating adheres to said surface-negative charged substance.

9. The apparatus according to claim 3 wherein the immobilizing means for immobilization of the mother cell comprises the mother cell coated with a coating of biotin, a plurality of avidin- or streptavidin-coated magnetic beads adhered to the coating of biotin and a magnet anchored to the plate.

10. The apparatus according to claim 3 wherein the immobilizing means for immobilization of the mother cell comprises the mother cell directly adhered to the plate using any one or combination of biotin, avidin or streptavidin.

11. The apparatus according to claim 3 wherein the immobilizing means for immobilization of the mother cell comprises the mother cell coated with a polyethylenimine coating and the at least one plate coated with a surface-negative charged substance wherein said polyethylenimine coating adheres to said surface-negative charged substance.

12. The apparatus according to claim 1 wherein the plate is an array of plates.

13. The apparatus according to claim 12 wherein the apparatus further comprises an actuator for engendering relative motion between the microscope and the array of plates such that the microscope can be positioned to individually observe each plate in said array of plates.

14. The apparatus according to claim 13 wherein the controller is further programmed to control the actuator.

15. The apparatus according to claim 1 wherein the microscope makes at least one observation of the plate.

16. The apparatus according to claim 1 wherein each observation is stored in a database.

17. The apparatus as claimed in claim 16 wherein each observation is compared to a predetermined threshold observation.

18. The apparatus according to claim 15 wherein each observation includes a plurality of periodic observations that can be analyzed by the controller to determine a replicative lifespan of the mother cell by counting a total number of offspring cells produced before senescence is reach by the mother cell.

19. The apparatus according to claim 1 wherein the microscope is a light microscope, a fluorescence microscope, a confocal microscope or an electron microscope.

20. The apparatus according to claim 1 wherein the at least one flow-through hole is configured to be approximately larger than a recently budded yeast cell.

21. The apparatus according to claim 1 wherein the at least one flow-through hole is larger than a mammalian cell.

22. The apparatus according to claim 1 wherein the mother cell is a stem cell, a progenitor cell or a yeast cell.

23. The apparatus according to claim 22 wherein the mother cell is any one of a Saccharomyces cerevisiae cell, a neuroblast, a mesenchymal stromal cell or a mesenchymal stem cell.

24. The apparatus according to claim 1 wherein the controller is programmed to determine if the mother cell divides either symmetrically or asymmetrically such that if the mother cell divides symmetrically the controller is programmed to begin observing both newly-created cells.

25. A method for quantifying replicative lifespan and observing senescence in cells, comprising:
preparing at least one plate,
immobilizing at least one mother cell capable of producing an offspring cell to the plate,
observing production of offspring cells by the mother cell with a microscope,
washing the plate with a wash fluid from a wash reservoir such that offspring cells are removed from proximity of the mother cell, and
determining the replicative lifespan of the mother cell by observing the total number of offspring cells produced by the mother cell.

26. The method as claimed in claim 25 comprising applying nocodazole to a progenitor cell resulting in a cell cycle block which produces the mother cell such that the mother cell is anchored to the progenitor cell.

27. The method as claimed in claim 26 wherein the progenitor cell is immobilized by coating the progenitor cell with biotin, adhering a plurality of avidin- or streptavidin-coated magnetic beads to the coating of biotin and anchoring a magnet to the plate.

28. The method as claimed in claim 26 wherein the progenitor cell is immobilized by adhering the progenitor cell to the plate using any one or combination of biotin, avidin or streptavidin.

29. The method as claimed in claim 26 wherein the progenitor cell is immobilized coating the progenitor cell with a polyethylenimine coating and treating the plate with a surface-negative charged substance wherein said polyethylenimine coating will adhere to said surface-negative charged substance.

30. The method as claimed in claim 25 wherein the immobilizing of the mother cell comprises coating the mother cell with a coating of biotin, adhering a plurality of avidin- or streptavidin-coated magnetic beads to the coating of biotin and anchoring a magnet to the plate.

31. The method as claimed in claim 25 wherein the immobilizing of the mother cell comprises a direct adhering of the mother cell to the plate using any one of biotin, avidin or streptavidin.

32. The method as claimed in claim 25 wherein the immobilizing of the mother cell comprises coating the mother cell with a polyethylenimine coating and treating the plate with a surface-negative charged substance wherein said polyethylenimine coating will adhere to said surface-negative charged substance.

33. The method according to claim 25 wherein a second observation is performed by the microscope after the washing of the plate to determine a quantity of remaining offspring cells that were not removed by washing in order to prevent counting the same offspring cell twice.

34. The method according to claim 25 wherein an actuator for engendering relative motion between the microscope and an array of plates can reposition the microscope such that the microscope can observe a plurality of plates.

35. The method according to claim 25 wherein a quantity of offspring cells produced by the mother cell between subsequent observations is stored in a database.

36. The method according to claim 25 wherein a quantity of periods where no offspring cells are produced by the mother cell is compared to a threshold quantity signaling that senescence has been reached by the mother cell.

37. The method as claimed in claim 36 wherein the threshold quantity represent three periods of observation.

38. The method according to claim 25 wherein the wash fluid exits the plate through a flow-through hole such that the at least one offspring cell can be removed from proximity to the mother cell.

39. The method according to claim 25 The method as claimed in any one of claims 25 to 38 wherein the microscope is a light microscope, a fluorescence microscope, a confocal microscope or an electron microscope.

40. The method according to claim 25 wherein the mother cell is a stem cell, a progenitor cell or a yeast cell.

41. The method as claimed in claim 40 wherein the mother cell is a Saccharomyces cerevisiae cell, a neuroblast, a mesenchymal stromal cell or a mesenchymal stem cell.

42. The method according to claim 25 wherein software is used to determine if the mother cell divided symmetrically or asymmetrically such that if the mother cell divides symmetrically the controller is programmed to begin observing both mother cells.
